(19) United States
(12) Patent Application Publication
(10) Pub. No.: US 2012/0189586 A1
(43) Pub. Date: Jul. 26, 2012

(54) Human Placental Derived Extracellular Matrix and Uses Therof

(76) Inventor: Carl Randall Harrell, Tarpon Springs, FL (US)

(21) Appl. No.: 13/354,964

(22) Filed: Jan. 20, 2012

Related U.S. Application Data
(60) Provisional application No. 61/461,689, filed on Jan. 21, 2011.
(60) Provisional application No. 61/461,680, filed on Jan. 21, 2011.

(51) Int. Cl. A61K 35/12; A61P 43/00; A61K 38/39

(52) U.S. Cl. 424/93.7; 514/17.2

(57) ABSTRACT

The invention provides a composition for use in tissue augmentation of a human. More specifically, the composition comprises collagen extracted from human placenta and a bulking agent. Furthermore, the invention discloses methods of augmenting soft tissue comprising the use of the compositions described herein.

### CROSS-REFERENCE TO RELATED APPLICATIONS

This nonprovisional application claims benefit of priority under 35 U.S.C. §119(e) of provisional application U.S. Ser. No. 61/461,689, filed Jan. 21, 2011, now abandoned, and provisional application U.S. Ser. No. 61/461,680, filed Jan. 21, 2011, now abandoned, both of which are hereby incorporated by reference in their entireties.

### BACKGROUND OF THE INVENTION

1. Field of the Invention

The present invention pertains to the field of uses collagen and adipose tissue. In particular, the invention concerns compositions and methods of using Type III based collagen in unique applications.

2. Background Art

Collagen, prepared from xenogenic (animal) sources such as bovine, porcine, and equine, has been used as a biomaterial for commercial applications involving extracellular matrix (1). Examples of products composed of animal collagen include dermal implants, wound dressings, tissues and biomaterials for hernia repair, bone graft substitutes, and artificial skin. The most abundant collagen is Type I collagen, accounting for most collagen in connective tissues in vertebrates. It is the only collagen that has seen significant commercial applications owing to its abundance and ease of purification. Type I collagen forms thick mechanically strong fibers found in connective tissues such as tendon and bone that require high mechanical forces. The second most abundant collagen is Type III collagen whose function is less well understood. Type III collagen co-localizes with Type I collagen in some tissues, but until now there have been very few studies of the tissue interaction when Type III collagen is implanted into tissues. Knock-out mice with no functional Type III collagen gene and no Type III collagen have a phenotype of fragile skin and aortas similar to the human connective disease of Ehlers-Danlos syndrome (2-3). Thus, Type III collagen may affect the fiber structure of Type I collagen (4). Type III collagen is present in much larger amounts in embryonic tissues and in early wound healing suggesting that Type III collagen form thin fibers that are more elastic than Type I collagen fibers (5). Fetal bovine dermis is about 18-30% Type III collagen, whereas adult dermis is typically less than 5% Type III collagen (6). Human fetal tissues were found to contain 18-21% Type III collagen and adult tissues were found to contain 8-11% Type III collagen (7).

Humallagen is a unique human collagen preparation consisting of Type I and Type III collagens isolated together from human placenta. Placentas contain approximately equal amounts of Type I and Type III collagens. Humallagen (Type I+Type III collagen) is purified from human placental tissue.

Bovine collagen has gained widespread use as an injectable material for soft tissue augmentation. The most immediate concern to most plastic surgeons is the fate of bovine collagen after injection. Zyderm I® with 35 mg/mL of collagen is rapidly degraded by tissue collagenases and resorbed within months. Zyderm II® with 65 mg/mL of collagen and, thus, almost twice the concentration of collagen, is longer lasting but follows the same fate as Zyderm I®. Zyplast® contains 35 mg/mL of collagen cross-linked with glutaraldehyde. Zyplast® is degraded over time. Patients receiving either Zyderm I® collagen, Zyderm II® collagen or Zyplast® collagen, (bovine collagen implants (BCI)), develop adverse immune reactions.

There remains a need for increasing the effectiveness of soft tissue augmentation wherein the biocompatible material has improved persistence, is not adverse to the patient and is readily available.

### SUMMARY OF THE INVENTION

The present invention is directed to a composition for augmenting tissue in a human, comprising a bulking agent and human collagen. In one aspect, the collagen is placental collagen. Preferably, the collagen comprises Type I and Type III collagen, wherein the Type III collagen is at least 30% of the volume of the collagen component. In one aspect, the composition further comprises a pharmaceutical excipient, an analgesic, a local anesthetic, an anti-inflammatory agent, an anti-microbial agent, a growth factor, a growth-promoting serum factor, or a combination thereof. Representative bulking agents include but are not limited to autologous globin, elastin, acellular human cadaveric dermis or autologous fibroblasts or a synthetic polymer. Representative synthetic polymers include but are not limited to polymethylmethacrylate microspheres, dextran beads, polylactic acid or silicones. In one form, the collagen is cross-linked and/or sterilized by gamma irradiation. Generally, the collagen is at least 1% of the composition volume. In one aspect, at least 50% of the composition comprises said bulking agent.

The present invention is further directed to a method to augment tissue in a human subject which method comprises administering the composition of the present invention to the tissue to be augmented in said subject. Representative tissues include but are not limited to breast tissue, sphincter tissue, buttocks tissue, fatty tissue, perineal body of the vagina, a cleft lip and corn tissue.

The present invention is further directed to a composition for contouring soft tissue in a subject comprising collagen and adipose tissue. The collagen may be placental collagen and for example, extracted from whole placenta. Typically, the collagen comprises Type I and Type III collagen, wherein the Type III collagen is at least 30% of the weight or volume of the collagen component. The collagen may be a mixture of human placental collagen and an additional collagen selected from the group consisting of recombinant human collagen, tissue engineered human-based collagen, autologous collagen, collagen fibers, and human tissue collagen matrix. Generally, the composition comprises 5-95% by weight of collagen and 5-95% by weight of adipose tissue. In one aspect, the composition comprises 30-70% by weight of collagen and 30-70% by weight of adipose tissue. The adipose tissue can be autologous. In one form, the composition further comprising a pharmaceutical excipient, an analgesic, a local anesthetic, an anti-inflammatory agent, or a combination thereof. In another form, the composition further comprises contractile tissue, subcutaneous tissue, dermal tissue, connective tissue, epidermal cells, or stem cells, or a combination thereof. In yet another form, the composition further comprises an anti-microbial agent, a growth factor, a growth-promoting serum factor, or a combination thereof.

The present invention is further directed to a method to contour soft tissue in a subject which method comprises applying the composition comprising collagen and adipose tissue into soft tissue and/or surrounding areas, so as to contour said tissue. Representative tissues include but are not limited to glabellar frown lines, nasolabial creases, circumoral wrinkles, sunken cheeks, a wrinkle, fold, depressions, scars, blemishes and scars. Typically, the composition administered is about 0.05-10.0 mL.

The present invention is further directed to a method of stimulating production of fibroblasts and adipocytes in a tissue of interest, comprising the step of contacting said tissue with a composition comprising Type I collagen and Type III collagen. In one aspect of this method, the composition is implanted into said tissue. Generally, the composition comprises about 25-50% Type I collagen and about 50-75% Type III collagen.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings.

These drawings form a part of the specification. It is to be noted, however, that the appended drawings illustrate preferred embodiments of the invention and therefore are not to be considered limiting in their scope.

FIGS. 1A-1C show results from implantation of human collagen into pig skin. FIG. 1A: Human collagen implanted in pig skin at one months. Significant inflammation occurred when heterologous collagen was implanted into pig skin. No inflammation was observed at one month when homologous procine collagen was implanted into pig skin and fibroblasts had actively migrated into the porcine collagen implant (not shown). FIG. 1B: When heterologous human collagen was implanted in pig skin at three months, significant and persistent inflammation occurred. FIG. 1C: When homologous porcine collagen was implanted in pig skin at three months, there was a lack of inflammation.

FIGS. 2A-2F show a nude rat study in which Humallagen was implanted subcutaneously in nude rats. Histology of the implant was obtained at 1, 2, and 3 months post implantation demonstrating the accumulation of fat cells in the implant. FIG. 2A: One month post implantation. Mag×200. FIG. 2B: One month post implantation. Mag×40.

FIG. 2C: Two (2) months post implantation. Mag×100. FIG. 2D: Two months post implantation. Mag×100. FIG. 2E: Three months post implantation. Mag×100. FIG. 2F: Three months post implantation. Mag×100. At three months, no material or tissue changes were observed with the exception of a higher number of fat cells infiltrating the gel material. New vessels of were formed within the gel material.

FIG. 3 shows an analysis at 1 month. Micrograph showing a section of the edge of the implant one month after implantation. Blood vessels have appeared at the periphery and within the implant along with many cells of unknown origin present in the spaces. Mag×400.

FIG. 4 shows a micrograph showing a section of the implant one month after implantation. A blood vessel is shown with two adipocytes adjacent to it. Mag×400.

FIG. 5 shows a micrograph showing a section of the implant two months after implantation. A blood vessel is surrounded by a large number of adipocytes. Mag×400.

FIG. 6 shows a micrograph showing a section of the implant one month after implantation. Adipocytes have apparently differentiated in rows within the spaces in the implant and not obviously adjacent to a blood vessel. Mag×400.

FIG. 7 shows a micrograph showing a section of the implant four weeks after implantation. Adipocytes early in their differentiation phase are present within spaces in the implant. These can be recognized as cells with spherical droplets in the cytoplasm. Mag×1000.

FIG. 8 shows a micrograph showing a section of the implant three months after implantation. After this time regions of the implants were almost entirely composed of mature adipocytes. Mag×400.

FIG. 9 shows adipose cells/unit area were quantified in the sections of tissue in the nude rat study as a function of time after implantation of Humallagen. Data obtained from 7-15 fields of view for each sample. Sample number at 4 weeks=13, number at 8 weeks=4, number at 8 weeks=6.

### DETAILED DESCRIPTION OF THE INVENTION

In one aspect, the invention provides a composition for augmenting tissue in a human subject which composition comprises a mixture of a bulking agent and human collagen. The composition may be injectable. In some cases, the collagen is placental collagen and may be from a single placenta. Providing compositions containing human collagen results in a reduced inflammatory response in a human.

In some embodiments, collagen comprises Type I and Type III collagen, wherein the Type III collagen is at least 30% of the weight or volume of the collagen component. The collagen may be cross-linked and/or sterilized by gamma irradiation, and may constitute at least 1% of the composition volume. The composition may further comprise a pharmaceutical excipient, an analgesic, a local anesthetic, an anti-inflammatory agent, an anti-microbial agent, a growth factor, a growth-promoting serum factor, or a combination thereof.

The bulking agent should be biocompatible and may comprise autologous globin and/or comprises a synthetic polymer. The synthetic polymer may comprise polymethylmethacrylate microspheres, dextran beads, polylactic acid and/or silicones. The bulking agent may also comprise elastin, acellular human cadaveric dermis or autologous fibroblasts or combinations thereof. In some embodiments, at least 50%, 60%, 75% or 90% of the composition comprises said bulking agent.

The invention also provides a method to augment tissue which method comprises administering the invention composition comprising human collagen and one or more bulking agent to the tissue to be augmented. The administering may be by injection. Representative tissue which can be augmented include breast tissue, buttocks tissue and fatty tissue. In some embodiments, said subject is afflicted with cachexia or AIDS wasting, or is afflicted with incontinence and said tissue is sphincter.

In another aspect, the invention also provides a method to augment tissue selected from the group consisting of the perineal body of the vagina, a cleft lip, sphincter, and corn tissue, which method consists of administering to said tissue a composition comprising human collagen. In some cases the collagen is derived from placenta, and in some cases from a single placenta. A bulking agent may be present, but is not necessary. In yet another aspect, the invention provides a method to facilitate fusion of a spinal disc space, which method consists of injecting into said tissue a composition comprising human placental collagen.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

A group of items linked with the conjunction “or” should not be read as requiring mutual exclusivity among that group, but rather should also be read as “and/or” unless expressly stated otherwise. Although items, elements, or components of the invention may be described or claimed in the singular, the plural is contemplated to be within the scope thereof unless limitation to the singular is explicitly stated.

### Collagen

Representative collagen materials include placental collagen, recombinant human collagen, tissue engineered human-based collagen, porcine collagen, bovine collagen, autologous collagen, collagen fibers, and human tissue collagen matrix. In certain embodiments, collagen in the compositions comprises collagen extracted from human placental tissue. Placental tissue includes the amnion and the chorion. The collagen may be extracted from pooled placental tissue, or from a single placenta. In certain embodiments, the collagen is extracted from the whole placenta. The collagen may be extracted according to disclosures in U.S. Pat. No. 5,002,071, which is incorporated by reference in its entirety herein. The collagen may be extracted by proteolytic extraction, for example, using pepsin, of any one or more of the following components: whole placenta insoluble amnion, soluble amnion, and soluble chorion of the placenta. Illustrative methods of preparing placental collagen for injectable compositions are described below.

Human sourced collagen is advantageous over compositions containing non-human sourced collagen, such as bovine collagen, when administered to humans. Allogeneic compositions exhibit a reduced immune response compared to injections of compositions containing material sourced from a different species than the recipient.

In certain embodiments, especially where repeated administration is needed, compositions comprising human placental collagen obtained from a single placenta are advantageous over collagen obtained from multiple or pooled placentas. Whereas pooled placenta may potentially introduce a greater diversity of potentially antigenic substances or foreign proteins, the preparation of collagen from a single placenta limits the diversity of these groups. Thus, injecting compositions comprising collagen from a single placenta introduces fewer potential antigens into the subject and reduces possible immune responses.

Type III collagen is present in rapidly growing tissue, particularly juvenile and healing skin. This is the collagen of granulation tissue and is produced quickly by young fibroblasts before the tougher Type I collagen is synthesized. Type III collagen has inter-chain disulfate bonds, whereas Type I collagen does not. The inter-chain disulfate bonds are one type of cross-linking and can provide additional molecular stability. An increase in cross-linked type collagen may result in a longer persistence of the collagen material when used in a subject as compared to lesser or no cross-linked materials. Cross-linked collagenous tissue as compared to lesser or no cross-linked tissue may have one or more of the following characteristics: increased tensile or structural strength, increased resistance to enzymatic degradation, reduced antigenicity, and reduced immunogenicity.

In certain embodiments, collagen having a high ratio of Type III collagen to Type I collagen may thus be particularly useful as they more closely mimic endogenous tissue. Injectable compositions containing Type III collagen may be useful in reducing the formation of excess scar tissue in wound healing by signaling to endogenous skin cells that there is sufficient scar tissue or young tissue already formed. Increasing the ratio of Type Ito Type III collagen may be useful to enhance the durability and strength of the injected composition.

Type III collagen may constitute at least 30%, 40%, 50%, 60%, 70%, 80%, 90%, or 95% of composition by volume of the collagen component. The collagen may comprise Type I and Type III collagen, wherein the Type III collagen is at least 30% of the weight or volume of the collagen component. In certain embodiments, the ratio of Type III to Type I collagen is equal to or greater than 30:70, 40:60, 45:55, 50:50, 55:45, 60:40, 65:35, 70:30, 75:25, 80:20, 85:15, 90:10, or 95:5. In certain embodiments, the ratio of Type III to Type I collagen is about 43:57, wherein “about” refers to ±15%. In one embodiment, Type I and Type II collagen are present in a 50:50 ratio.

The human collagen may include recombinant human collagen, tissue engineered human-based collagen, autologous collagen, collagen fibers, and human tissue collagen matrix, collagen fibers, or human tissue collagen matrix derived from cadaveric dermis.

Collagen may be treated to increase the level of crosslinking present as compared to the untreated form, for example, by heat, gamma irradiation, or contact with a synthetic or natural crosslinking agent. A non-limiting example of a crosslinking agent is glutaraldehyde, polyethylene glycol, or formaldehyde. In certain embodiments, only Type I or Type III collagen is subjected to further crosslinking prior to use in a tissue augmentation procedure. One or both of Type I or Type III collagen may be crosslinked. For example, in some compositions, Type I collagen is crosslinked and Type III collagen is crosslinked, or Type I is non-crosslinked and Type III collagen is non-crosslinked, or Type I collagen is crosslinked and Type III collagen is non-crosslinked, or Type I collagen is non-crosslinked and Type III collagen is crosslinked.

In certain embodiments, the collagen is cross-linked and/or sterilized by gamma irradiation. The collagen in the injectable compositions may be treated by terminal sterilization according to disclosures in U.S. Publication No. 2006/0280769. In specific embodiments, this method further comprises freezing the collagen material and irradiating the collagen material with an effective amount of gamma or electron beam radiation to sterilize the collagen material without causing significant deterioration of the collagen material. Gamma ray or e-beam radiation is at least 5 kGy, or between 6 kGy and 8 kGy. In some embodiments, the collagen is sterilized prior to contact with the bulking agent. In other embodiments, the collagen, after sterilization, is stored and handled under sterile conditions prior to injection into a subject.

The major molecular species besides collagen that are found in the extracellular matrix include the noncollagenous structural glycoproteins, elastin, and proteoglycans.

Collagen may be provided in pure and/or crystalline form to eliminate the noncollagenous proteins, which may be antigenic. Once the inflammatory cycle is stimulated, the resorption of collagen occurs by the infiltrating inflammatory cells, principally macrophages and, to a lesser extent, granulocytes. These cells contain collagenase which acts to digest collagen. Skin collagen is chemotactic itself and becomes even more active by digestion with tissue collagenase into smaller peptide fragments. Chemotropism is the attraction of living protoplasm to chemical stimuli whereby the cells are attracted (positive chemotaxis) or repelled (negative chemotaxis) by acids, alkalis or other bodies exhibiting chemical properties.

Various types of collagens, their alpha-chains, as well as small peptides formed by collagenase digestion have been shown to be chemotactic to dermal fibroblasts. Chemotactic migration of fibroblasts into the site of tissue injury or theoretically injected collagen can be regulated by the solubilized collagen or its degradation products. Thus, a collagen implant would not remain dormant in the tissue, but a complex series of events may occur.

Analysis of the physical properties of collagen can be carried out by methods known to a person having ordinary skill in the art. A non-limiting example includes the use of differential scanning calorimetry (DSC) can be used to evaluate collagen material. Comparison of shifts in melting temperature before and after irradiation, for example, provides information on the nature of the material including phase transitions.

### Bulking Agents

Compositions of the present invention comprise human collagen and a bulking agent. In certain embodiments, the bulking agent comprises globin, in particular, autologous globin, and/or a synthetic polymer. Representative synthetic polymers include but are not limited to polymethylmethacrylate microspheres, dextran beads, polylactic acid, silicones, polyethylene glycol (PEG), polyoxyethylene, polymethylene glycol, polytrimethylene glycols, polyvinylpyrrolidones, polyoxyethylene-polyoxypropylene block polymers and copolymers, and derivatives thereof. The bulking agent may also comprise elastin, acellular human cadaveric dermis and/or autologous fibroblasts. Other polymers that may be used in the composition include naturally occurring polymers such as proteins, starch, cellulose, heparin, hyaluronic acid, and derivatives thereof.

Further specific examples of bulking agents include acellular human cadaveric dermis that has been freeze-dried and micronized, globin (the protein portion of hemoglobin), and cultured autologous fibroblasts. Non-animal derived materials include dextran beads suspended in hylan gel of non-animal origin, polylactic acid, silicones made of man-made polymers in the form of solids, gels, or liquids as a function of polymerization and cross-linkage, expanded polytetrafluoroethylene (e-PTFE) for facial plastic and reconstructive surgery, in the form of sheets, strips, and tubes. Bulking agents also include compositions for soft tissue augmentation disclosed in U.S. Pat. No. 6,231,613 to Greff, et al., which are polymers having a water equilibrium content of less than about 15%. Exemplary polymers include cellulose acetates, ethylene vinyl alcohol copolymers polyalkyl (C1-C6) acrylates, acrylate copolymers, and polyalkyl alkacrylates wherein the alkyl and the alkyl groups contain no more than 6 carbon atoms. Other bulking agents include commercially available materials, for example, Restylane, Juvederm, FG-5017 from Fibrogen; and ISOLAGEN.

Collagen is at least 1% by volume of the compositions of the invention; or may be at least 5%, 10%, 15%, 25%, 50%, 75% or 90% of the composition volume. The bulking agent is at least 5%, 10%, 15%, 25%, 50%, 75% or 90% of the composition volume. Thus, collagen may be in an amount from 5% to 90% by volume of the composition and the bulking agent is in an amount from 10% to 95% by volume, or collagen is in an amount from 2% to 50% by volume and the bulking agent is in an amount from 50% to 98% by volume, or collagen is in an amount from 2% to 30% by volume and the bulking agent is in an amount from 70% to 98% by volume, or collagen is in an amount from 1% to 20% by volume and the bulking agent is in an amount from 80% to 99% by volume. In certain embodiments, the composition further comprises at least one or more of the following agents: a pharmaceutical excipient, an antimicrobial agent, an analgesic, a local anesthetic, an anti-inflammatory agent, and a growth factor. Non-limiting examples of antimicrobial agents include iodine, penicillin, silver compounds, sulfonamides, and erythromycin.

Non-limiting examples of local anesthetics include lidocaine, xylocaine, benzocaine, chloroprocaine, cocaine, cyclomethycaine, dimethocaine/larocaine, propoxycaine, procaine/novocaine, proparacaine, tetracaine/amethocaine, articaine, bupivacaine, carticaine, cinchocaine/dibucaine, etidocaine, levobupivacaine, lidocaine/lignocaine, mepivacaine, piperocaine, prilocaine, ropivacainean, trimecaine, and any combination thereof. Non-limiting examples of anti-inflammatory agents are ibuprofen, aspirin, naproxen, and glucocorticoids. Non-limiting examples of growth factors include transforming growth factor beta (TGF-_), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), and hepatocyte growth factor (HGF).

Pharmaceutical excipients include carriers suitable for parenteral administration. Examples of carriers are saline, buffered saline, dextrose, water, and other physiologically compatible solutions such as Hank's solution, Ringer's solution, or physiologically buffered saline. Penetrants appropriate to the particular barrier to be permeated are used in the formulation. Such penetrants are generally known in the art. For preparations comprising proteins, the formulation can include stabilizing materials, such as polyols, e.g., sucrose, and/or surfactants, e.g., nonionic surfactants, and the like. Alternatively, formulations for parenteral use can comprise dispersions or suspensions of the components of the compositions prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, or dextran. Optionally, the suspension also can contain suitable stabilizers. Emulsions, e.g., oil-in-water and water-in-oil dispersions, also can be used, optionally stabilized by an emulsifying agent or dispersant, i.e., surface active materials or surfactants. Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

In certain embodiments, the antimicrobial agent, analgesic, local anesthetic, anti-inflammatory agent, and/or growth factor are combined with the collagen prior to contact with the bulking agent. In alternative embodiments, the antimicrobial agent, analgesic, local anesthetic, anti-inflammatory agent, and/or growth factor is combined with the collagen after contact of the collagen with the bulking agent.

In certain embodiments, the collagen formulation is 35 mg/mL collagen in solution, wherein the solution is isotonic relative to the recipient host tissue. In some embodiments, the collagen is combined with a bulking agent, wherein the collagen, prior to combination with the bulking agent, has any concentration range in solution. In certain embodiments, the concentration range of collagen in solution is 5-50 mg/mL which does not have to be in solution. A collagen solution can be prepared with any of the pharmaceutical excipient carriers described herein.

The compositions may also further comprise biological tissue, such as adipose tissue, contractile tissue, subcutaneous tissue, dermal tissue, connective tissue, or a combination thereof. The biological tissue may be autologous.

The composition may further comprise at least one eukaryotic cell type, such as keratinocytes, stem cells, fibroblasts, melanocytes, adipocytes, T-cells, or combinations thereof. The eukaryotic cells may increase the structural integrity of connective tissue, promote healing, and/or promote the integration of the composition with the recipient site. Furthermore, the eukaryotic cell, such as a fibroblast, may enhance or promote the growth or connection of cells or tissues.

### Methods of Tissue Augmentation

The methods may use the compositions of the invention that comprise human collagen and a bulking agent for applications that require administering relatively large amounts of augmenting material, or may simply employ human collagen without bulking agent where small amounts are needed. Applications which employ the invention compositions containing bulking agents include enlargement or reconstruction of breasts, filling out of buttocks, and the like. Applications where a bulking agent is not required include vaginal tightening, repair of cleft lip, effecting of fusion of spinal disc space, and the like. Some applications could employ human collagen wither with or without bulking agent depending on the severity of the condition. These include repair of the urethral or anal sphincter. The appropriate composition for use in a particular form of tissue augmentation will be apparent to the practitioner.

The human collagen compositions, with or without bulking agent, may be administered to selected tissues by any practical means, such as direct injection, application into an incision, or topical application. In many cases, injection is most practical, but other methods of administration may also be employed.

Methods to augment tissue in human subjects employing a composition comprising human collagen and a bulking agent may target any area of the body where the augmentation is desirable. Such uses include, but are not limited to, buttocks, breast or fatty tissue. In general, any area which experiences loss of fat tissue between the skin and muscle over time can benefit from the compositions and methods of tissue augmentation described herein. Such areas occur in subjects afflicted with cachexia or AIDS wasting.

Thus, the composition and methods can be used for breast augmentation. Breast tissue augmentation can be used to correct deformities like a mastectomy, a breast implant collapse and or a tuberous breast, a condition in which the adult breasts fail to develop in puberty and result in extremely small, narrow and sagging breasts. The composition can be used while contained in a shell or without a shell so long as the composition material remains in a particular area.

The compositions and methods described herein may be used to fill out the buttocks in a procedure known as gluteoplasty. In one embodiment, small incisions are made in each gluteal cheek, and the composition is injected at various levels in the subject's buttocks. Each deposit of the composition may be less than 5 cc and in some instances, the amount is equivalent to the size of a single pearl or pea. When a bulking agent comprising cellular material such as adipose tissue is used, the size or volume of the composition in each deposit is small enough to encourage the development of a blood supply to the newly transferred tissue.

The composition may also be injected into the hip tissue area and is known as ‘hip augmentation’ or ‘hip enlargement’, for instance in a subject desiring a more pronounced curve of the hips, or the composition can be used to augment tissue of the calves as well as fatty tissue in the body. The administration can be into any position in the specific area such as epidermis, dermis, fat, or subcutaneous layer. In one embodiment, the composition may be injected into a section of skin of the human and deposited beneath the surface of the skin, such as within or near the epidermis and/or dermis layers of the skin.

The compositions of the invention are useful in subjects when it is necessary to compress the urethra to assist the sphincter muscle in closing to avoid leakage of urine from the bladder. The invention provides a composition which can be used to add bulk and localize compression to the sphincter muscle/urethra, thereby reducing the lumen size and/or increasing support to the sphincter/urethra through one or more injections of the augmentation material described herein.

The administration of the collagen containing composition described herein makes the area around the urethra thicker, which helps control urine leakage. It can also improve the lost support of the bladder and urethra. Thus, stress incontinence due to incompetent sphincters in females and males can be substantially reduced. Subjects who can benefit from a periurethral injection with composition described herein may have stress incontinence as a result of pregnancy, childbirth, aging, or damage by scarring from surgery or radiotherapy.

Methods to augment tissue that do not require bulking agent include augmentation of the perineal body of the vagina, of a cleft lip, of sphincter and of corn tissue. One method comprises injecting into said tissue a composition comprising human collagen, e.g., human placental collagen. The invention also provides a method to facilitate fusion of a spinal disc space, by injecting into said space a composition comprising human collagen. In some cases, the human collagen comprises placental collagen. In some cases, less than a total of 20 cc is injected into the tissue or spinal disc space.

Vaginal tightening is often desired in women who have experienced childbirth. Human collagen compositions, especially human placental compositions, may be used to remedy this by supplying the composition to the perineal body, typically in a single administration, typically by direct injection. Appropriate administration techniques are illustrated in the examples below.

The cleft lip may exist in a subject as a small gap or an indentation in the lip (partial or incomplete cleft) or continues into the nose (complete cleft). Lip cleft can occur as one sided (unilateral) or two sided (bilateral). A mild form of a cleft lip is a microform cleft. This can appear as small as a little dent in the lip or look like a scar from the lip up to the nostril. Generally, injection of small amounts of human collagen composition, e.g., less than 10 cc, is effective.

In some embodiments, the administration is repeated over time until a desired total amount of composition is delivered; in other cases only a single administration event is required. The amount of the injected composition is related to the desired amount of enlargement in a particular area and the capacity of the area to be treated. The amount may be related to the desired resulting aesthetic of the enlarged area, or to the desired function of the enlarged area. In other embodiments, the method comprises at least one administration to the area of desired enlargement during one sitting with the practitioner, or can be spaced out over time and multiple visits to the practitioner. In some embodiments, the methods of the invention comprise the use of micro injection, wherein small amounts of injectable composition is administered into a site. For example, a single injected amount of the composition may be 0.01-10.0 cc, or 0.01-1.0 cc or 0.01-0.1 cc.

Multiple injections may be required in an augmentation procedure. The total amount injected into a tissue may be at least 10 cc, 15 cc, 20 cc, 30 cc, 50 cc, 75 cc, 100 cc, 150 cc, 200 cc, or 500 cc. In some cases, an excess of composition volume is injected to compensate for any absorption of the composition by the body which may decrease the long-term volume. In some cases, an excess of composition volume is not required to compensate for any absorption of the composition by the body because of the composition's high persistence in the body. In certain embodiments, less than a total of 50 cc, 40 cc, or 20 cc of the composition is injected into the tissue. In certain embodiments, the amount of the composition injected into the section of soft tissue and/or surrounding area is equivalent to the amount of desired enlargement. The total amounts depend on the tissue selected for augmentation, the desired appearance of the tissue resulting from the augmentation, and/or the amount needed to carry out a particular function, such as correcting incontinence. For example, a breast augmentation may require upwards a total of 100 cc or more, whereas a periurethral injection may require about 14 cc of the composition.

In certain embodiments, the total amount of the composition injected into the section of soft tissue is equivalent to the amount that will change the appearance of the soft tissue and/or the physical characteristics of the soft tissue, such as size, shape, firmness, softness, and location. A skilled practitioner would appreciate the teaching of the present invention as a whole and be able to determine the exact amount and frequency of injection for each particular case.

Any mode of administration that results in the desired outcome may be employed. For application of the compositions of the invention which contain both human collagen and a bulking agent, and relatively large amounts are needed, it may be desired to prepare an incision and deposit the composition into the cavity created. Alternatively, the compositions may be employed topically. For relatively small amounts, in particular, it may be desirable to administer the compositions by direct injection.

Injection can be carried out by syringe, catheters, needles, and other means for injecting or infusing the injectable composition. For example, injection may be carried out by injecting through a needle of 25 gauge. The lumen size of the delivery device should be large enough to permit the transfer of the composition into the body without degradation of the material, yet small enough to reduce the size of the incision or opening into the body.

The frequency and the amount of administration such as by injection under the present invention are determined based on the nature and location of the particular are of augmentation desired. The stable and long lasting character of the present invention compositions may result in multiple injection not being necessary or being reduced. A skilled practitioner should be able to determine the frequency and the amount of the injection for each particular case.

The injection method of the present invention can be carried out by any type of sterile needle and corresponding syringe or other means for injection, such as a three-way syringe. The needles, syringes and other means for injection are commercially available from suppliers such as VWR Scientific Products (West Chester, Pa.), Beckton Dickinson, Kendal, and Baxter Healthcare. The size of the syringe and the length of the needle used will depend on the particular injection based on factors such as the specific disease or disorders being treated, the location and depth of the injection, and the volume and specific composition of the injectable suspension being used. In certain embodiments, the needle gauge is 17, 25 or 30. A skilled practitioner will be able to make the selection of syringe and needle based on experience and the teaching of the present invention.

In some cases, the injectable composition and/or methods of augmenting soft tissue described herein stimulate the formation of adipocytes and/or adipocyte deposition in a subject compared to compositions and methods that do not incorporate the use of the human or placental collagen described herein. In some embodiments, the injectable compositions and/or methods of tissue augmentation described herein promote vascularization in a subject compared to compositions and methods that do not incorporate the use of the human or placental collagen described herein. The compositions and methods described herein may also promote fibrovascular growth in a subject into the injectable composition, and results in a better “take” and reduced rejection of the injected composition and are non-immunogenic.

In one aspect, the invention provides a composition for contouring soft tissue in a subject comprising (a) collagen; (b) adipose tissue; and (c) optionally a pharmaceutical excipient, an analgesic, a local anesthetic, an anti-inflammatory agent, or a combination thereof. The collagen may be from a human source such as placental collagen. The collagen is extracted from a whole placenta. The composition has a reduced inflammatory response in a human subject compared to compositions containing collagen from a non-human source.

In certain embodiments, the collagen is extracted by proteolytic digestion of a source selected from the group consisting of insoluble amnion, soluble amnion, soluble chorion of the placenta and combinations thereof. The collagen comprises Type I and Type III collagen. In more particular embodiments, the Type III collagen is at least 30% of the weight or volume of the collagen component.

In certain embodiments, the collagen is a mixture of collagens from at least two distinct tissue sources. In some embodiments, one of the collagens is placental collagen and the other collagen material(s) is selected from the group consisting of recombinant human collagen, tissue engineered human-based collagen, autologous collagen, collagen fibers, and human tissue collagen matrix. The collagen may also be a commercial collagen product (e.g. recombinant human collagen type III FG-5017 from Fibrogen; or ISOLAGEN.) The compositions may further comprise a pharmaceutically acceptable excipient.

In certain embodiments, the adipose tissue of the composition is autologous. In certain embodiments, the collagen is homogenized to pass through a 30 gauge surgical needle. In certain embodiments, the composition is injectable. In certain embodiments, the collagen is from soluble amnion of the placenta. In certain embodiments, the collagen is cross-linked and/or sterilized by gamma irradiation. In certain embodiments, the composition comprises an analgesic, a local anesthetic, an anti-inflammatory agent, or a combination thereof. In certain embodiments, the proteolytic digestion is with pepsin. In certain embodiments, the collagen is at least 1% of the composition volume. In certain embodiments, the composition further comprises an anti-microbial agent, a growth factor, a growth-promoting serum factor, or a combination thereof. In some embodiments, the growth factor is TGF-β.

In another aspect, the invention provides a method to contour soft tissue in a subject which method comprises injecting or otherwise applying a composition of the invention described herein into a selected section of soft tissue and/or surrounding areas, whereby said soft tissue is contoured. In certain embodiments, the method is used to treat a subject with glabellar frown lines, nasolabial creases, circumoral wrinkles, sunken cheeks, and/or depressed scars. In certain embodiments, the composition is deposited within or near the epidermis and/or dermis layers of the skin. In more particular embodiments, the section of the skin is located on the hands, feet, face, or neck. In certain embodiments, the selected section of soft tissue is a wrinkle, fold, depression, scar, or blemish. In certain embodiments, said composition is injected with a surgical needle with a gauge of 30.

In certain embodiments, the amount of the composition administered is about 0.05-10.0 mL. In certain embodiments, the amount of the composition administered in a single injection is about 0.05-10.0 mL.

In certain embodiments, the method of the invention further comprises treating the selected section of soft tissue with microdermabrasion, chemical peels, BOTOX cosmetic injections, or Lipodissolve, prior to or after said injecting.

In certain embodiments, injecting the composition containing human collagen results in a reduced inflammatory response in a human subject compared to compositions containing collagen from a non-human source. In certain embodiments, the injected composition described herein persists for a longer period of time in the host tissue compared to adipose-containing compositions that do not comprise human placental collagen.

In certain embodiments, the collagen is a mixture of collagens from two distinct tissue sources. In certain embodiments, the second collagen material is selected from the group consisting of recombinant human collagen, tissue engineered human-based collagen, autologous collagen, collagen fibers, and human tissue collagen matrix.

In certain embodiments, the ratio of Type III to Type I collagen is equal to or greater than 30:70, 40:60, 45:55, 50:50, 55:45, 60:40, 65:35, 70:30, 75:25, 80:20, 85:15, 90:10, or 95:5 by weight or volume. In certain embodiments, the ratio of Type III to Type I collagen is about 43:57, wherein “about” refers to +15%. In one embodiment, Type I and Type III collagen are present in a 50%:50% ratio.

In some embodiments, the collagen concentration is prepared as 35 mg/mL in a solution that is isotonic relative to the adipose and/or recipient host tissue. In some embodiments, the collagen is combined with adipose tissue, wherein the collagen, prior to combination with adipose, has any concentration range in solution. In certain embodiments, the concentration range of collagen in solution is 5-50 mg/mL. In some embodiments, the collagen is not in solution. A collagen solution can be prepared with any of the pharmaceutical excipient carriers described herein.

In another embodiment, the collagenous tissue is further treated to increase the level of crosslinking present as compared to the untreated form. In one embodiment, increasing the level of crosslinking is achieved by heat, gamma irradiation, or contact with a synthetic or natural crosslinking agent. A non-limiting example of a crosslinking agent is glutaraldehyde, polyethylene glycol, or formaldehyde. In certain embodiments, only Type I or Type III collagen is subjected to further crosslinking prior to use in a tissue contouring procedure.

In another embodiment, either one or both of Type I or Type III collagen may be crosslinked. In another embodiment, Type I collagen is crosslinked and Type III collagen is crosslinked. In another embodiment, Type I is non-crosslinked and Type III collagen is non-crosslinked. In another embodiment, Type I collagen is crosslinked and Type III collagen is non-crosslinked. In another embodiment, Type I collagen is non-crosslinked and Type III collagen is crosslinked.

When using collagen as a biomaterial, it is important to use it in its purest and crystalline form to eliminate the noncollagenous proteins that are far more potent antigens. Once the inflammatory cycle is stimulated, the resorption of collagen occurs by the infiltrating inflammatory cells, principally macrophages and, to a lesser extent, granulocytes. These cells contain collagenase which acts to digest collagen. Skin collagen is chemotactic itself and becomes even more active by digestion with tissue collagenase into smaller peptide fragments. Chemotropism is the attraction of living protoplasm to chemical stimuli whereby the cells are attracted (positive hemotaxis) or repelled (negative chemotaxis) by acids, alkalis or other bodies exhibiting chemical properties. Various types of collagens, their alpha-chains, as well as small peptides formed by collagenase digestion have been shown to be chemotactic to dermal fibroblasts. Chemotactic migration of fibroblasts into the site of tissue injury or theoretically injected collagen can be regulated by the solubilized collagen or its degradation products.

Thus, a collagen implant would not remain dormant in the tissue, but a complex series of events may occur. First, the collagen implant could be invaded by inflammatory and fibroblasts and, while being continuously resorbed, it could promote an inflammatory reaction by chemotactic properties of its degradation products. Thus, the area of collagen metabolism is not only important for collagen and other soft tissue injectable materials, but also to both normal and abnormal wound healing, i.e. hypertrophic scarring and keloids. In certain embodiments, the collagen is substantially free of noncollagenous proteins.

Any type of adipose tissue may be used in the composition. Methods to harvest adipose tissue are described in the art. In some embodiments, the adipose tissue is obtained from the lower abdomen, inside or outside of the thighs, back, hip, and buttocks. In certain embodiments, the adipose tissue is autologous.

In certain embodiments, the composition comprises 5-95% by weight of collagen and 5-95% by weight of adipose tissue. In certain embodiments, the composition comprises 5-90% by weight of collagen and 10-95% by weight of adipose tissue. In certain embodiments, the composition comprises 30-70% by weight of collagen and 30-70% by weight of adipose tissue. In certain embodiments, the composition comprises 2-50% by weight of collagen and 50-98% by weight of adipose tissue. In certain embodiments, the composition comprises 2-30% by weight of collagen and 70-98% by weight of adipose tissue. In certain embodiments, the composition comprises 1-20% by weight of collagen and 80-99% by weight of adipose tissue.

In certain embodiments, the composition further comprises contractile tissue, subcutaneous tissue, dermal tissue, connective tissue, epidermal cells, or stem cells, or a combination thereof.

The composition and methods of the invention are used to contour soft tissue. In certain embodiments, methods of the invention are applied to treat glabellar frown lines, deep nasolabial creases, circumoral wrinkles, sunken cheeks, frown lines, worry lines, wrinkles, crow's feet, marionette lines, stretch marks, depressed scars, and internal and external scars including scars resulted from injury, wounds, accidents, bites, and surgery, stretch marks on various positions of the skin, especially on the stomach, areas of the lower body, and legs after weight loss and/or child bearing; and a sunken and abnormally rounded eyelid after an upper or lower lid blepharoplasty, wherein excessive fat has been removed. In certain embodiments, the composition is administered to any of the areas described to these areas described.

In certain embodiments, tissue contouring can be administered to an area of the body where the contouring is desirable. The composition can be administered to any area of the subject's body wherein treatment is desired, including, but not limited to, face, neck, torso, arms, hands, legs, and feet. For example, two areas in the face that seem to show the effects of fat loss the earliest include the area around the mouth and the area around the eyes. Tissue contouring around the brow re-creates the softer appearance of a youthful eyelid. The large buccal fat pad that spreads throughout the cheeks and around the facial muscles also dissipate over time and are areas where tissue contouring is desirable. In another specific embodiment, areas that are subject to the methods of the invention include the cheeks, nose, lips, forehead, neck, and the areas around the eyes. In general, any area which experiences loss of fat tissue between the skin and muscle over time is an area which can benefit from the compositions and methods of tissue contouring described herein.

In certain embodiments, the composition and/or methods of contouring soft tissue described herein stimulate the formation of adipocytes and/or adipocyte deposition in a subject compared to compositions and methods that do not incorporate the use of the placental collagen described herein. The compositions and/or methods of tissue contouring described herein promote vascularization in a subject compared to compositions and methods that do not incorporate the use of the placental collagen described herein.

In certain embodiments, selected soft tissue is epidermis, dermis, fat or subcutaneous layer and the composition is injected into epidermis, dermis, fat, or subcutaneous layer. In an embodiment, the administration of the composition is directed to the subcutaneous layer. In certain embodiments, the inventive method is to be used to fill in fine lines and wrinkles, especially in the face.

The compositions and methods, as described and utilized herein, are used for orthopaedic tissues, such as tendons or ligaments, or nerve tissue and/or to improve wound healing at these tissue sites. These compositions are effective to augment repair and reinforcement of these tissues.

The composition and methods of the invention are used to stimulate production of fibroblasts and adipocytes in a tissue of interest. In certain embodiments the tissue of interest is contacted with a composition comprising Type I collagen and Type III collagen. In certain aspects the Type I collagen may be about 25-50% of the composition. In other aspects the Type III collagen may be 50-75% of the composition. The tissue of interest is contacted via implantation or injection of the collagen composition.

The present invention additionally provides a kit for contouring soft tissue. The kit comprises a 30 gauge or finer needle and a corresponding syringe, wherein the syringe contains an injectable composition described herein. The needle, syringe and composition are sterile and ready to use. The kits are designed in various forms based on the sizes of the syringe and the needles and the volume of the injectable composition contained therein, which in turn are based on the specific skin deficiencies the kits are designed to treat. In certain embodiments, the amount of composition in the syringe is between 0.1-2.0 mL in volume. In other embodiments, the volume is between 0.1 and 5.0 mL.

### Manufacture of Placental Collagen

In making the soft injectable material, fresh placenta is collected and the amnion is manually separated from the chorion, such as by finger separation. Both the amnion and the chorion are then cleaned of any remaining blood clots or debris. For short-term storage, the amnion and the chorion are placed in an antibiotic solution until processed. Exemplary antibiotic solutions include linomycin (3 gms/10 mL), amphotericin B (50 mg/10 mL), neomycin sulfate (0.5 gm/10 mL), polymyxin B sulfate (500,000 units/10 mL) in 1 liter of normal saline.

Collagen is extracted using limited proteolytic digestion with pepsin. In brief, tissue is homogenized in 0.5 M acetic acid, the pH adjusted to 2.5 with HCl and the preparation digested twice with pepsin (10 mg pepsin/gm wet weight tissue) overnight. A combination method of selective precipitation from neutral salt solvent and acid solvents is used to purify the collagen. Purified collagen is reconstituted by dialysis against low ionic strength sodium phosphate buffer (pH 7.2) at 15-17° C. Lidocaine was added to a final concentration of 0.3%. All procedures are carried out at 4-8° C., although other temperatures can be used. In addition, an anti-microbial agent, an anti-inflammatory agent, a growth factor, or a combination thereof is optionally incorporated after reconstitution of the collagen, depending on the desired properties of the collagen layer.

### Insoluble Amnion Processing

The following steps are taken to extract collagen from the amnion of the placenta. The amnion is stored in an antibiotic solution according to Example 1. First, the antibiotic is decanted from the amnion. Then, 5 mL of cold distilled water is added to each amnion, with subsequent homogenization of the amnion for approximately 15 minutes in polytron. The homogenized amnion is then centrifuged at 8,000×g for 15 minutes at 4° C. The supernatant is then discarded and the precipitant washed five times with acetone to remove the lipids. The precipitant is then weighed, and pepsin (Sigma, 1:10,000, from porcine stomach mucosa) 3.0 molar acetic acid per amnion was added, 15 mL or more if extra large amnions, and the precipitant is homogenized for approximately 5 minutes in a polytron. The mixture is allowed to stand for 18 hours at 4° C., centrifuged at 100,000×g for 1 hour at 4° C., the supernatant discarded, the precipitant weighed and the pepsin and homogenization steps are repeated and the supernatant discarded. In addition, an anti-microbial agent, an anti-inflammatory agent, a local analgesic, a growth factor or a combination thereof can be added to the precipitant after the supernatant is discarded and mixed, depending on the desired properties of the collagen layer.

### Soluble Amnion Processing

A presently preferred way of processing soluble amnions from the placenta comprises rinsing the antibiotics from the amnions with deionized water, adding 5 mL of cold distilled water to each amnion, homogenizing for approximately 15 minutes in a polytron and centrifuging at 8,000×g for 15 minutes at 4° C. The supernatant is discarded and lipids are removed from the precipitate by washing with acetone three times and weighing the precipitate.

Pepsin (Sigma, 1:10,000, from porcine stomach mucosa) is added to the precipitate (1:100 w/w) and 100 mL of 0.5 molar acetic acid per amnion is added, more if the amnions are extra large, and then homogenized for approximately 10 minutes in a polytron. The pepsin is allowed to extract collagen from the precipitate for 18 hours at 4° C. and then centrifuged at 100,000×g for 1 hour at 4° C. retaining both the precipitate and the supernatant. The supernatant is again weighed, and the steps of pepsin and acetic acid addition, homogenization, pepsin extraction of collagen and centrifuging are then repeated.

The supernatants from the first and second extractions are combined and 10-molar NaOH is added drop wise to adjust the pH to from 7.0 to 7.2. The mixture is permitted to stand for 2 hours at 4° C., centrifuged at 100,000×g for 45 minutes at 4° C. and the precipitate is discarded. A 3.0M NaCl solution is added to the supernatant and permitted to stand for 2 hours at 4° C., centrifuged at 100,000×g for 45 minutes at 4° C. and the precipitate is weighed and lidocaine to 0.3% is added.

Soluble Amnion Processing with Further Purification

A presently preferred method of soluble amnion processing from the placenta and further purification comprises rinsing the antibiotic from the amnion with deionized water, the amnions are cut to approximately 2 cm×2 cm and washed briefly with acetone, soaked in 0.5 M acetic acid (pH adjusted to 2.5 with HCl), homogenized with a polytron for about 15 minutes, pepsin is added (1:100 pepsin/set tissue) (1 mg pepsin/1 mL solution) and stirred at 4° C. overnight, centrifuged as indicated above, retaining the supernatant. Pepsin is again added to the mixture as indicated previously and stirred at 4° C. overnight, centrifuged and the supernatant from both centrifuging steps are combined. A 2.0M NaCl solution is added to the mixture and permitted to stand overnight at 4° C. and again centrifuged, the supernatant discarded and the precipitate retained.

The precipitate is purified by dissolving it in 0.5 M acetic acid, centrifuging, and discarding the precipitate. A 2.0M NaCl solution is added to the supernatant, and permitted to stand overnight at 4° C., again centrifuged with the supernatant discarded. The resulting precipitate is dissolved in 0.5 M acetic acid, again centrifuged, and the precipitate discarded. The supernatant is dialysed against 0.02 M Na₂HPO₄ thoroughly for 48 hours with frequent dialysis fluid exchanges, centrifuged, the supernatant discarded, the precipitate weighed, and solid lidocaine HCl is added to 0.30% with mechanical agitation.

### Chorion Processing

In a presently preferred method of processing soluble chorion, the antibiotics are rinsed from the chorion with deionized water, the chorion is cut to approximately 2 cm×2 cm units and washed briefly with acetone and then soaked into 0.5 M acetic acid that had been adjusted to pH 2.5 with HCl. The tissue is then homogenized with a polytron to fine particles for about 15 minutes, pepsin added and centrifuged as indicated above with the supernatant being retained. The pepsin and centrifuge steps are then repeated, the supernatant of each of these steps are combined with 2M NaCl and permitted to stand overnight at 4° C. and then centrifuged again with the supernatant discarded.

For purification, the precipitate is dissolved into 0.5 M acetic acid, centrifuged, and the precipitate discarded. A 2M NaCl solution is added to the supernatant and permitted to stand overnight at 4° C., then again centrifuged and the supernatant discarded. The precipitate is dissolved into 0.5 M acetic acid, centrifuged, dialysed against 0.02 M Na₂HPO₄ thoroughly for 48 hours with frequent dialysis fluid exchanges, again centrifuged, the supernatant discarded, and the precipitate weighed. Solid lidocaine HCl is added to 0.30% to the precipitate with mechanical agitation.

### Cross-Linking and Sterilizing

Collagen precipitates obtained by any of the foregoing preparations are treated with a radioactive source, wherein the material is sterilized and cross-linked. 15 cc of each of the foregoing resulting precipitates is placed in 20 cc serum bottles with crimp closures and placed in cesium-137 radioactive source for varying lengths of time in order for them to receive 0.25 M rads, 0.5 M rads, 1.0 M rads, and 2.0 M rads which serves the dual purpose of sterilizing the material and cross-linking the collagen.

### Sterilizing

Collagen precipitates obtained by any of the foregoing preparations is treated with a radioactive source, wherein the material is sterilized as described in U.S. Pat. No. 7,902,145. A 0.3% to 0.5% human Type I+III collagen solution was prepared at pH 3 (lower than 5), filtered through a 0.45 μm porous membrane, and then processed under a laminar flow hood in a class 1000 clean room. No bacteria were detectable in the filtered solution. The collagen is precipitated by addition of 20 mM sodium phosphate, at pH 7.2, at room temperature. The collagen paste was harvested by centrifugation in closed and sterile buckets. The 6% concentrated collagen paste was then washed and diluted to 3.5% with a sterile phosphate buffered physiological solution (PBS). Sterile 1 mL syringes were filled with the final collagen paste. After one week of storage at +4° C., each syringe was packed within its final pouch and sealed before being frozen in dry ice to about −80° C. Each layer of syringes was covered by a one inch thick layer of dry ice, within an insulated polystyrene box. The total height of the final package was less than 15 inches and it was stored at −20° C. or in dry ice until gamma-irradiation. Gamma-irradiation was performed at room temperature for less than 24 hours. The irradiation dose was >25 kGray. Some dry ice was still present in the package after irradiation and the syringes were still frozen. After thawing, the syringes were inspected. The syringes were not damaged and they were stored at room temperature for one week before being tested. The collagen paste was tested using Sodium Dodecyl Sulfate-Poly Acrylamide Gel Electrophoresis (SDS-PAGE) procedures well known to one of ordinary skill in the art.

The following examples serve to describe more fully the manner of using the above-described invention, as well as to set forth the best modes contemplated for carrying out various aspects of the invention. It is understood that these examples do not serve to limit the true scope of this invention, but rather are presented for illustrative purposes.

### Example 1

### Preparation of Injectable Composition Containing Collagen and Bulking Agent

Human placental collagen was extracted as described above. However, collagen obtained by any of the preparations A-E may be used. The collagen is extracted from a single placenta and is sterilized. In this procedure, the collagen layer is frozen at a temperature of −20° C. Then, the collagen is treated with 8 kGy of radiation, resulting in a sterility assurance level of 10⁻⁶ SAL for the collagen and then thawed to room temperature.

The sterilized collagen is formulated in an amount of 35 mg/mL collagen in saline solution and is combined and homogenized with the bulking agent comprising biocompatible polymethylmethacrylate microspheres. The mixture is prepared such that the composition can pass through a 17 gauge needle. The composition comprises about 10% of the sterilized collagen and about 90% of polymethylmethacrylate. A total volume of about 100 cc is prepared.

### Example 2

### Breast Tissue Augmentation

The composition prepared in Example 1 was injected into layers of the breast through six to eight, two millimeter incisions in each breast. Blunt syringes and cannulas are used to inject the composition so that no damage is done to blood vessels or nerves. The composition is layered from the pectoralis major muscle up through the top of the breast.

Injection of the composition is carried out to shape the breast or breasts for an aesthetic, natural-looking result. The amount of each injection is between 0.1-1.0 cc. The composition is injected into the breast tissue to create the desired shape until the desired amount is transferred into the selected region.

### Example 3

### Tissue Augmentation Around the Urethra

This example describes correction of urine incontinence by two different routes. For the periurethral route, the composition described in Example 1 is dispensed into syringe connected to a 17-gauge needle. If the density of the composition requires, a special high-pressure injector may be used to inject the dense composition into the periurethral tissues. The needle is slowly inserted next to the urethral opening and into the submucosal tissues. After ascertaining the proper position of the needle, the composition is injected at 3 places around the urethra: the 2-, 6-, and 10-o'clock positions. As the injection progresses, the urethral lumen can be observed closing, and then the opening disappears. To assure success, complete apposition of the urethral mucosa at the end of the procedure is observed. One or 2 tubes of 7 cc of the composition may be injected to produce complete closure of the urethra. For the transurethral route, the a syringe, the composition is injected under direct vision underneath the urethral mucosa. A cystoscope is inserted into the mid urethra. Under cystoscopic vision, the tip of the needle is inserted precisely underneath the urethral mucosa. The composition is carefully injected into the submucosal tissues until complete coaptation of the urethral mucosa is visualized.

### Example 4

### Tissue Augmentation of the Vagina

This example describes augmentation of tissues relating to the vagina, in particular vaginal tightening subsequent to childbirth. A composition comprising human placental collagen is dispensed into syringe connected to a 20-gauge needle. The needle is inserted into the perineal body. After ascertaining the proper position of the needle, the composition is injected at various depths. The perineal wall and muscles are provided with support from the injected composition, and vaginal tightening is achieved. Optionally, additional injections adjacent to the posterior and anterior vaginal walls, exterior to the vaginal canal are carried out such that the vaginal walls are provided with additional support. The result of the procedure includes additional support of the perineum muscles, a decrease in internal and external diameters, a decrease in vaginal stretching, and an overall tightening of the vagina. This procedure restores the vagina to its status before pregnancy.

### Example 5

### Tissue Augmentation to Repair a Cleft Lip

This example describes tissue augmentation of a cleft lip in a child. This procedure closes an incomplete cleft, wherein the cleft is an abnormal indention in the lip, and is carried out when the subject is 6 to 12 weeks old. While the subject is under general anesthesia, a composition comprising human placental collagen is dispensed into syringe connected to a 20-gauge needle. The composition is injected into the indentation and the tissue surrounding the indentation. Multiple injections may be required and the total amount of the composition is injected is an amount that fills in the indentation. This procedure restores the lip to a continuous and uninterrupted state.

### Example 6

### Preparation of Injectable Adipose Containing Composition

In an alternative example, the ratio of the collagen/adipose ratio is adjusted such that the homogenized mixture can be administered using a 30 gauge needle or smaller bore needle.

### Example 7

### Preparation of an Injectable Adipose-Collagen Containing Composition

The collagen preparation is mixed with a suspension containing 60 mg/mL of adipose tissue in saline. The ratio of collagen to adipose tissue is 9:1 by volume.

### Example 8

Injection of Collagen Adipose Composition into a Mammal

Approximately 1 mL of the injectable composition prepared in Example 1 is filled into the barrel of a 1 mL syringe attached to a 30 gauge needle. An area of desired soft tissue contouring, such as a depressed region on the cheek of a human subject, is identified. The composition is injected into the depressed region until the desired amount is transferred into the selected region.

### Example 9

### Preparation of a Collagen-Adipose Composition

The procedure above is modified so that a dense consistency of the composition is obtained by not adding saline solution to the collagen and freshly harvested adipose tissue. This composition can be administered to the selected tissue by opening the skin, depositing the composition into said opening, and suturing the opening.

### Example 10

### Materials and Methods

Type I collagen was purified from pig dermis according to methods for purification of Type I collagen from bovine dermis. Humallagen (the mixture of Type I+Type III human collagen) was purified from human placentas according to the methods described by (8-9). When Type I collagen itself was required, it was separated from Type III collagen by salt precipitation. The placentas were washed to eliminate blood components. The placental tissue was digested by pepsin in an acidic solution. Types I, I+III and IV collagens were separated by selective increase of the NaCl concentration. The absence of residual pepsin was checked by a specific ELISA method. The collagen fractions were dried in acetone and the respective powders were stored frozen.

### Example 11

### Pig Study

The purpose of this study was to compare homologous Type I collagen (isolated from porcine skin) with heterologous Type I collagen (isolated from human placenta) injected into pig dermis. Porcine and human highly purified collagens of the same purity and concentration were implanted in the dermis of domestic pigs (Seghers hybrid). Homologous collagen was Type I collagen purified from pig skin, and heterologous collagen was human Type I collagen purified from human placenta. The concentration of collagen was 35 mg/mL. Pigs were injected subcutaneously with 0.3 mL human collagen or porcine collagen. They were followed for seven days, one month, three and five months. At each time point, biopsies were obtained; the tissue including the implant was embedded, sectioned, stained and analyzed by histology. The tissues were stained with either trichrome or H and E staining and examined by light microscopy.

### Nude Rat Study

The purpose of this study was to characterize the tissue response to Humallagen injected subcutaneously into nude rats. Nude rats (Rnu/Rnu) were used in order to avoid an immunological reaction to human collagen in the rat. Humallagen was injected subcutaneously and the animals were followed for up to three months. Nude rats were obtained from Charles River and housed until used. Ten rats were divided into three groups and were each injected subcutaneously with 0.1 mL Humallagen, 35 mg/mL. They were housed separately and exsanguinated at three time points of one, two and three months post implantation. At each time point the tissue containing the implant was obtained by gross dissection and fixed in neutral buffered formalin. Following dehydration and embedding in paraffin wax, sections were cut on a Leica microtome at 10 mm. Following dewaxing in xylene, rehydration through a graded series of alcohols and rinsing in phosphate buffered saline (PBS), sections were stained using the Gomori Trichrome method with a kit from Richard-Allen Scientific, dehydrated and mounted with Cytoseal (Thermo Scientific).

### Results

Humallagen was characterized by SDS electrophoresis for purity and for the weight fraction of Type I and Type III human collagens. The material was >99% purified collagen and consisted of 48% Type 1 and 52% Type III human collagens.

### Pig Study

Pigs were injected subcutaneously with Type I porcine collagen or Type I human collagen on day zero. Tissues were examined histologically at seven days, and no difference was observed between porcine and human collagens. No inflammation was observed for both implants, demonstrating the excellent short-term tolerance of both implants and the absence of any visible short term immune reaction. Animals sacrificed at one, three and five months and examined histologically were observed to have major differences in the porcine tissue reaction observed for porcine and human collagen implants.

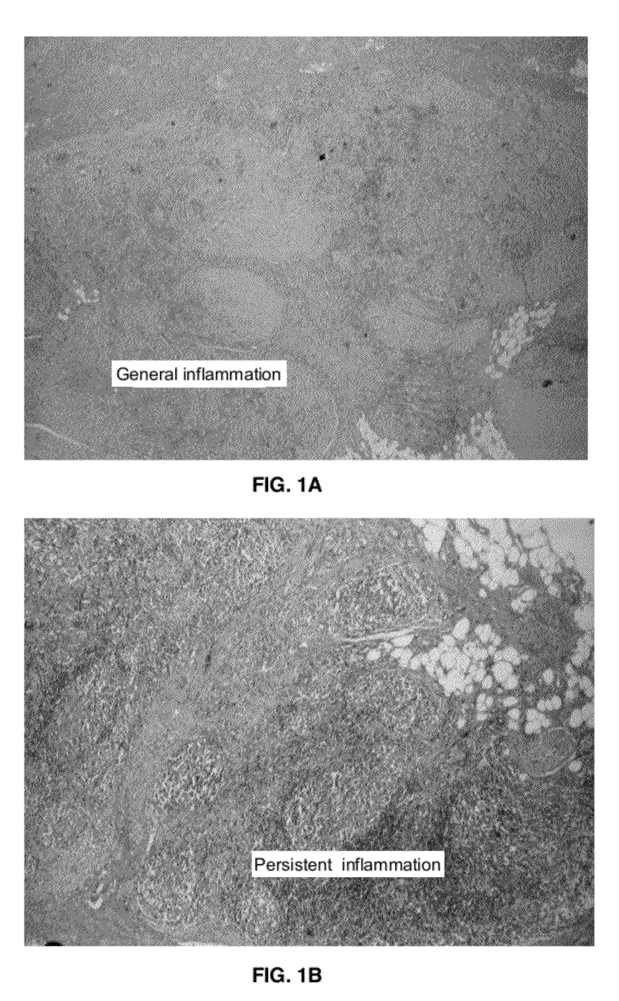

At three months, there was a strong and persistent inflammation induced by the human collagen in pig dermis with numerous lymphocytes, monocytes, and granulocytes demonstrating a long-term inflammatory reaction (FIGS. 1A-1B). In contrast to the result with a human collagen implant, the porcine collagen implant in pig skin did not induce any inflammation, and there was a migration of fibroblasts synthesizing collagen in the porcine dermis (not shown). The local environment of inflammatory reactions induced by a heterologous collagen negatively interfered with fibroblasts synthesizing new collagen. In the animals with the homologous implant, there is an early migration of active fibroblasts starting their local collagen synthesis in a quiet environment (FIG. 1C). This observation is not unexpected since bovine collagen injected in human dermis is associated with inflammation. Previous reports (10) have shown that 3% of humans demonstrate an immunological hypersensitivity reaction.

### Nude Rat Study

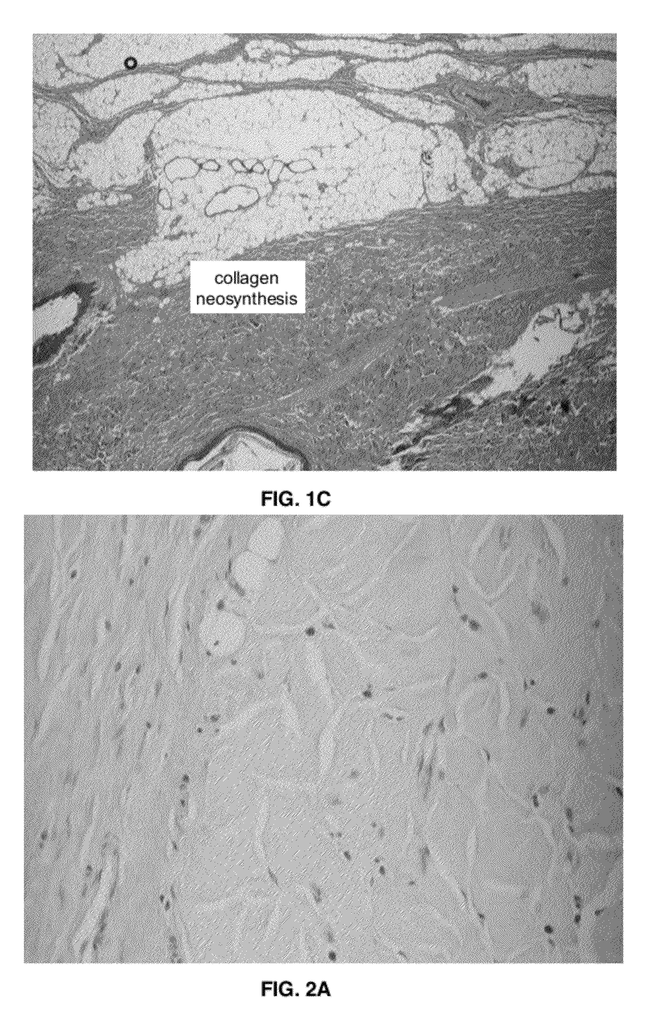

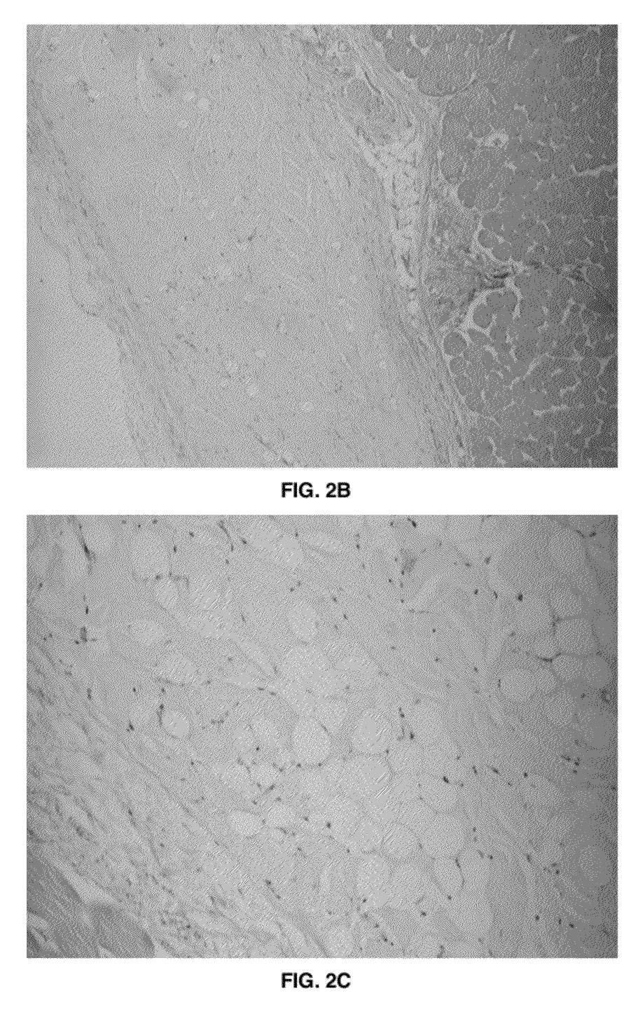

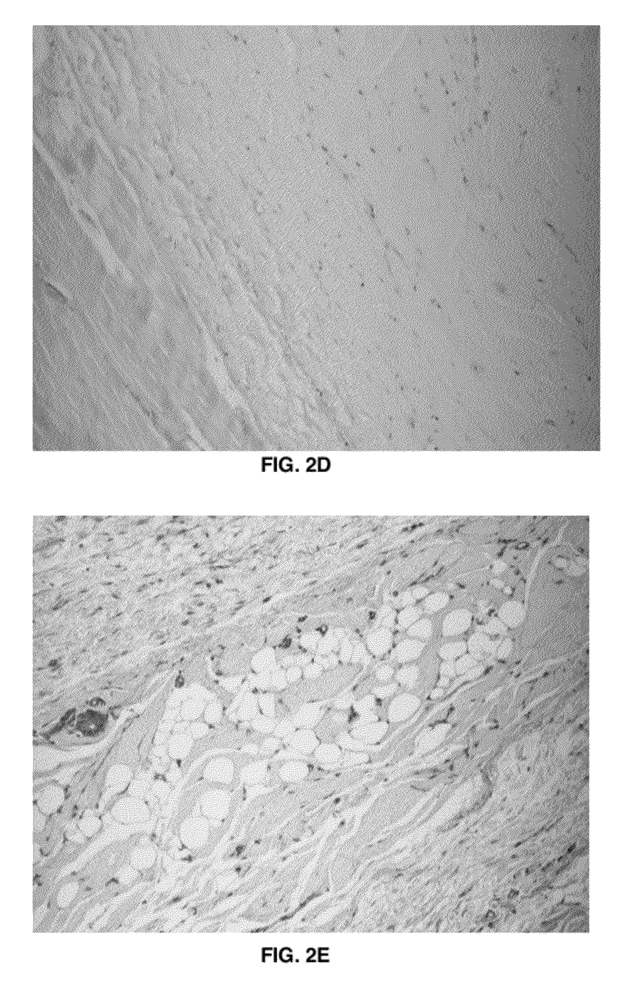

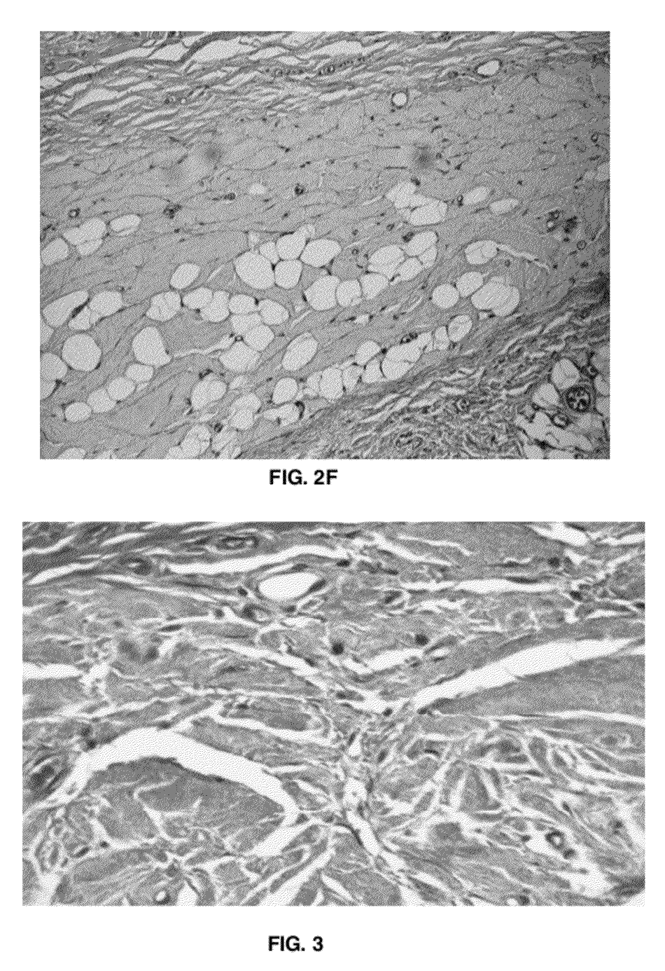

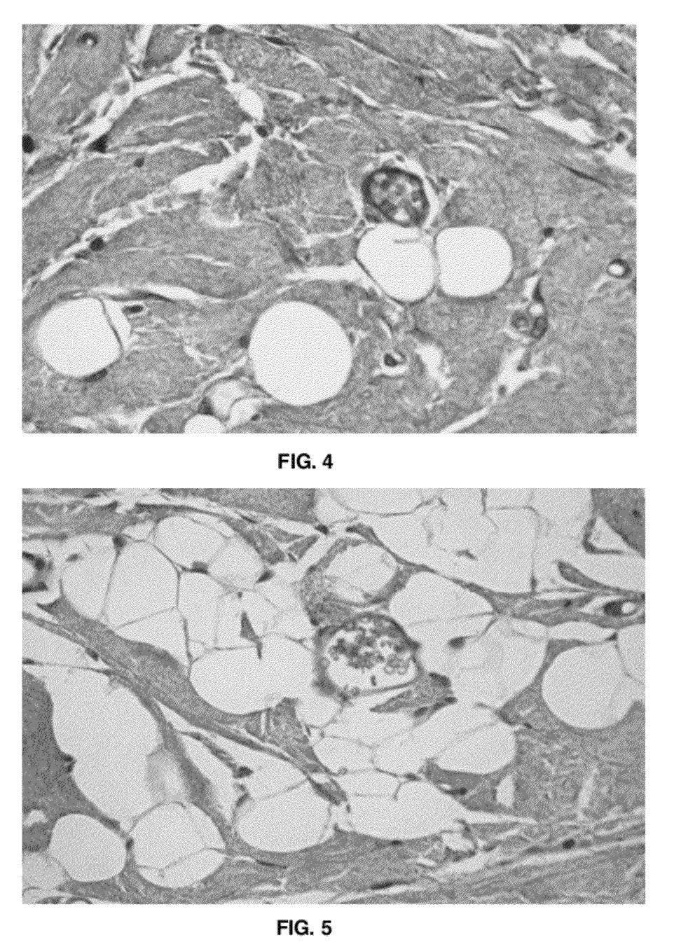

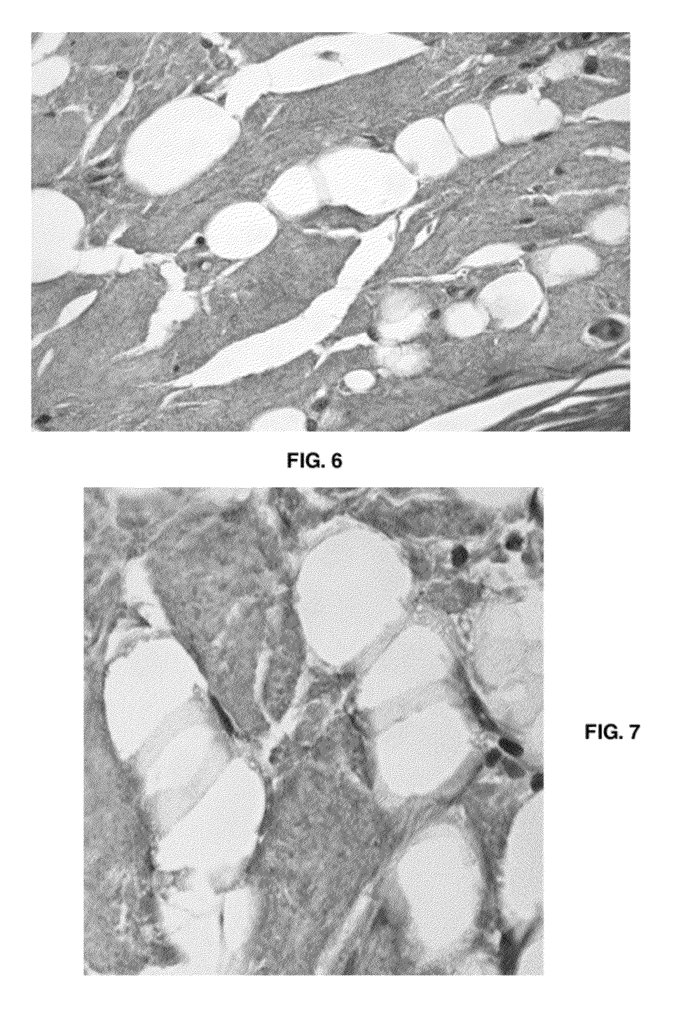

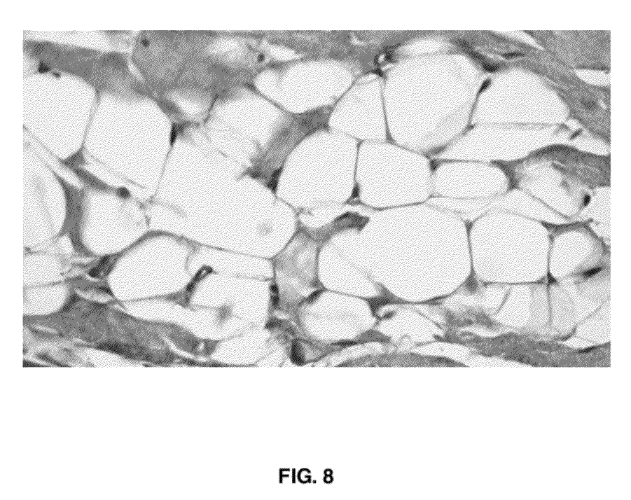

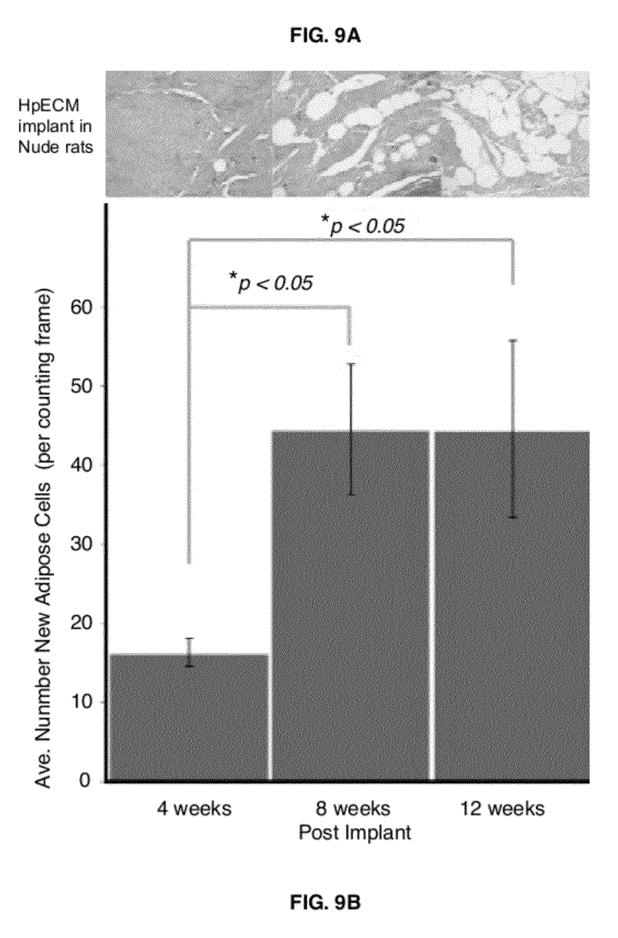

When human collagen was injected subcutaneously into nude rats there was no inflammatory reaction as expected since the nude rat is immunologically compromised. At three months the histological analysis was quite dramatic in that the implanted Humallagen became vascularized and spaces in the Humallagen implant became filled with fibroblasts and expressed abundant levels of adipocytes (FIG. 2). The origin of these adipocytes could be either differentiation of fibroblasts into adipocytes or the proliferation of adipocyte stem cells. Since there is very little connective tissue adjacent to the Humallagen as it was placed subcutaneously, and in most cases was placed below the panniculus muscle, an adipocyte stem cell source is more likely. One further possibility for their origin is hematopoietic stem cells brought in by the vascularization. Support for this idea is that the Humallagen implant first became vascularized, the spaces within the implant became infiltrated with cells (FIG. 3) and the initially differentiating adipose cells were often observed to be adjacent to the blood vessels (FIG. 4). Eventually large, localized areas of adipose cells were observed around a blood vessel (FIG. 5). However not all adipocytes differentiated around blood vessels as the cells which initially appeared in spaces (FIG. 3) also seemed to differentiate thereby producing transverse lines of adipocytes (FIG. 6). Many of these cells could be seen early in their differentiation phase when their cytoplasm still contained fat droplets (FIG. 7). After three months the implants had area within them entirely composed of mature adipocytes (FIG. 8). These histology slides were used to measure the number of adipose cells per unit area of the implant, and the data were tabulated for all of the sections examined at each time point. When the area of the field covered by adipose cells was examined for each time point, there is a highly significant difference between the quantity of adipose cells at 4 and 8 weeks (p<0.05), as seen in FIG. 9. There is no significant difference in the number of adipose cells between 8 weeks and 12 weeks.

### Discussion

The function of Type III collagen in tissues is not clearly understood. Type III collagen is found with type I collagen in several tissues but predominantly in skin and vascular tissue. Humans having Ehlers-Danlos Syndrome have abnormal Type III collagen owing to mutations in Type III collagen genes. They are identified by having fragile skin and fragile vessels especially a mechanically weak aorta that may rupture and cause death.

In order to understand the role of Type III collagen an animal model was created in which Type III collagen genes were inactivated. Homozygous mice (Col3−/−) missing both genes for Type III collagen mostly died in utero or within 48 hours after birth. The few who survive had disorganized skin and aortas subject to rupture (5) consistent with Ehlers-Danlos Syndrome. Heterozygous (Col3+/−) mice having approximately half of the normal amount of Type III collagen were found to have reduced wound closure rates and increased scar formation compared with wild type mice (11).

Another approach to understanding the role of Type III collagen in tissue is to examine tissues that have substantial amounts of Type III collagen. Fetal tissues have two to six times more Type III collagen in skin than in an adult expressed as an increased percentage of Type III collagen compared with Type I collagen (6). One significant observation with fetal skin is that in contrast to adults, fetal skin also heals with little scarring. Although several suggestions regarding growth factor composition have been made to explain this observation that fetal skin heals with minimal or no scarring (12) a role for Type III collagen in contributing to reduced scar formation may be applicable especially since mice expressing low amounts of Type III collagen were observed to have slower healing and reduced scarring (11).

The present invention shows that the presence of Type III collagen in an implant is associated with an increase of adipocytes in the implant. The observation supporting this result is illustrated in FIG. 9 where it can be seen that the accumulation of fat cells at two and three months is dramatic. In a rat model of soft tissue augmentation (13), it was observed that Type I+Type III human collagen injected into the skin of rats revealed fibroplasia, vascular infiltration, and the development of adipocytes with the implant as well as a lack of inflammatory response following up to 12 months of implantation. A preparation of human Type I+Type III collagens purified from human placentas was implanted in rats subcutaneously (14). In this study human collagen injected into rats persisted for 12 months and was not associated with any significant inflammatory reaction. Blood vessels were seen on the surface of the implant as early as 30 days and the amount of newly synthesized collagen and blood vessels accumulated from one to six months.

Several clinical studies have been performed using human collagen for implantation into the dermis. A safety study was performed using Type I human collagen in humans in a population of subjects that are allergic to bovine collagen, and those patients sensitive to bovine collagen did not react to human collagen (9). A clinical study testing the performance of human collagen for dermal augmentation was carried out in China (14). The test material was human Type I+Type III collagen purified from placentas. In this study 123 subjects were enrolled in the study for six months duration, and a subgroup of 30 subjects was followed for a long term of up to two years. Each patient received two injections 30 days apart in either the frontal furrow, the glabellar lines in the forehead or crows' feet. The effects lasted 8 to 9 months after the second injection.

A safety study was performed using Humallagen for dermal augmentation in the US as an IDE pilot study (15). The study involved 25 subjects. The study design was a single site study, with a double blind split face design on the nasolabial fold. One side of the face was treated with Humallagen and the contra-lateral side was treated with a control injection of Cosmoderm human Type I collagen. The study period was six months. The results supported the safety and performance of Humallagen compared with Cosmoderm. The results also confirmed that there was no detectable immunological reaction to either of the two human collagen preparations using a skin test injection of 0.1 mL into the volar forearm surface examined at three days and 28 days post injection. This is in contrast to the multiple decade experience with bovine collagen injections where it has been observed that up to 3% of the population is allergic to bovine collagen injection. There were no significant differences between Humallagen and Cosmoderm in terms of adverse events, or effectiveness.

The present invention demonstrates that the Humallagen preparation examined here triggers the formation of fat tissue at the implant site. The presence of significance amounts of Type III collagen in Humallagen is responsible for the increased adipocyte recruitment and differentiation to adipose tissue. There may also be an additional effect of Type III collagen in reducing scar formation. The hypothesis that Type III collagen is responsible for an increase in adipocytes or a decrease in scarring is tested in an animal model where Humallagen consisting of Type I and Type III human collagens are compared with human Type I collagen alone for the recruitment of adipocytes and the reduction in scar formation when implanted in dermal wounds.

The following references are cited herein.
1. Miyata et al. Clinical materials, 9(3-4):139-148 (1992).
2. Kuivaniemi et al. Jour of Biological Chem, 265(20):12067-12074 (1990).
3. Kontusaari et al. Amer Journal of Human Genetics, 51(3):497-507 (1992).
4. Romanic et al. Jour of Biological Chem, 266(19):12703-12709 (1991).
5. Liu et al. Proceedings of the National Academy of Sciences of the United States of America, 94(5):1852-1856 (1997).
6. Ramshaw, J. A. Connective Tissue Research, 14(4):307-314 (1986).
7. Smith et al. The American Journal of Anatomy, 175(4):507-521(1986).
8. Tiollier et al. Exp Cell Res, 191(1):95-104 (1990).
9. Toman, et al. Plast Reconstr Surg, 113(3):1015-1020 (2004).
10. DeLustro et al. Plast Reconstr Surg, 79(4):581-594 (1987).
11. Volk et al. Cells, Tissues, Organs. (2011)
12. Ferguson and O'Kane. Philosophical Transactions of the Royal Society of London. Series B, Biological sciences 359(1445):839-850 (2004).
13. Spira, M., B. Liu, et al. J Biomed Mater Res, 28(1):91-96 (1994).
14. Liu, B., Z. Xu, et al. Seminars in Plastic Surgery, 19:241-250 (2005).
15. Albiorex (2009). Pilot Investigational Study to Compare Injectable Humallagen™ to Cosmoderm™ for the Treatment of Nasolabial Folds.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A composition for augmenting tissue in a human, comprising a bulking agent and human collagen.

2. The composition of claim 1, wherein the collagen is placental collagen.

3. The composition of claim 1, wherein the collagen comprises Type I and Type III collagen, wherein the Type III collagen is at least 30% of the volume of the collagen component.

4. The composition of claim 1, further comprising a pharmaceutical excipient, an analgesic, a local anesthetic, an anti-inflammatory agent, an anti-microbial agent, a growth factor, a growth-promoting serum factor, or a combination thereof.

5. The composition of claim 1, wherein the bulking agent comprises autologous globin, elastin, acellular human cadaveric dermis or autologous fibroblasts or a synthetic polymer.

6. The composition of claim 5, wherein the synthetic polymer comprises polymethylmethacrylate microspheres, dextran beads, polylactic acid or silicones.

7. The composition of claim 1, wherein the collagen is cross-linked and/or sterilized by gamma irradiation.

8. The composition of claim 1, wherein the collagen is at least 1% of the composition volume.

9. The composition of claim 1, wherein at least 50% of the composition comprises said bulking agent.

10. A method to augment tissue in a human subject which method comprises administering the composition of claim 1 to the tissue to be augmented in said subject.

11. The method of claim 10, said tissue is selected from the group consisting of breast tissue, sphincter tissue, buttocks tissue, fatty tissue, perineal body of the vagina, a cleft lip and corn tissue.

12. A composition for contouring soft tissue in a subject comprising collagen and adipose tissue.

13. The composition of claim 12, wherein the collagen is placental collagen.

14. The composition of claim 13, wherein the collagen is extracted from whole placenta.

15. The composition of claim 12, wherein the collagen comprises Type I and Type III collagen, wherein the Type III collagen is at least 30% of the weight or volume of the collagen component.

16. The composition of claim 15, wherein the collagen is a mixture of human placental collagen and an additional collagen selected from the group consisting of recombinant human collagen, tissue engineered human-based collagen, autologous collagen, collagen fibers, and human tissue collagen matrix.

17. The composition of claim 12, which comprises 5-95% by weight of collagen and 5-95% by weight of adipose tissue.

18. The composition of claim 12, which comprises 30-70% by weight of collagen and 30-70% by weight of adipose tissue.

19. The composition of claim 12, wherein the adipose tissue is autologous.

20. The composition of claim 12, further comprising a pharmaceutical excipient, an analgesic, a local anesthetic, an anti-inflammatory agent, or a combination thereof.

21. The composition of claim 12, further comprising contractile tissue, subcutaneous tissue, dermal tissue, connective tissue, epidermal cells, or stem cells, or a combination thereof.

22. The composition of claim 12, further comprising an anti-microbial agent, a growth factor, a growth-promoting serum factor, or a combination thereof.

23. A method to contour soft tissue in a subject which method comprises applying the composition of claim 12 into soft tissue and/or surrounding areas, so as to contour said tissue.

24. The method of claim 23, wherein said tissue is selected from the group consisting of glabellar frown lines, nasolabial creases, circumoral wrinkles, sunken cheeks, a wrinkle, a fold, a depression, a scar, a blemish and a scar.

25. The method of claim 23, wherein the composition administered is about 0.05-10.0 mL.

26. A method of stimulating production of fibroblasts and adipocytes in a tissue of interest, comprising the step of:
contacting said tissue with a composition comprising Type I collagen and Type III collagen.

27. The method of claim 26, wherein said composition is implanted into said tissue.

28. The method of claim 26, wherein said composition comprises about 25-50 percent by weight Type I collagen.

29. The method of claim 26, wherein said composition comprises about 50-75 Type III percent by weight collagen.
